(19) United States
(12) Patent Application Publication
(10) Pub. No.: US 2020/0411152 A1
(43) Pub. Date: Dec. 31, 2020

(54) Increasing Return of Unused Controlled Substance Prescriptions

(71) Applicant: CERNER INNOVATION, INC., Kansas City, KS (US)

(72) Inventors: Girish Gangadhara, Bengaluru (IN); R. Madhu Sudhan, Bengaluru (IN); Alan H. Staples, II, Shawnee, KS (US)

(21) Appl. No.: 16/572,788

(22) Filed: Sep. 17, 2019

(30) Foreign Application Priority Data
Jun. 28, 2019 (IN) 201941025993

(51) Int. Cl. G16H 20/10; G16H 15/00; G16H 10/60; G06F 9/54; H04L 12/58; H04W 4/14

(52) U.S. Cl. CPC G16H 20/10; G16H 15/00; H04W 4/14; G06F 9/542; H04L 51/38; G16H 10/60

(57) ABSTRACT

Technologies are provided for dynamically engaging with individuals, family members, guardians, and/or caretakers to increase the return of unused controlled substances to one or more collection centers. After receiving an indication that a first medication has been prescribed and that the medication is a controlled substance, various sets of data are identified to determine one or more collection centers within a predetermined distance from the individual for return of unused medications. Additionally, a stop date for the first medication is determined and a message is transmitted to the individual on a user interface to remind the individual of a stop date for the medicine and to return unused medications to the designated collection centers.

### CROSS-REFERENCE TO RELATED APPLICATIONS

This application, having attorney docket number 27098.327491 and entitled “Increasing Return of Unused Controlled Substances,” claims priority to India Provisional Patent Application No. 201941025993, filed Jun. 28, 2019, and entitled, “Increasing Return of Unused Controlled Substances,” the entirety of which is incorporated here by reference.

### BACKGROUND

Addiction to controlled substances, such as opioids, has reached record levels over the last several years. Addiction is a primary, chronic disease of brain reward, motivation, memory, and related phenomena. Abnormality in these functions leads to characteristic biological, psychological, social, and spiritual manifestations. This is reflected in an individual pathologically pursuing reward or relief by substance use and other behaviors. Often times, individuals may be prescribed controlled substances for medically appropriate reasons, but do not properly dispose of unused medications, thereby creating opportunity for misuse by the individual or others who might have access to these unused medications.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

In most cases, addiction to controlled substances begins with a medically valid and legitimately acquired prescription to a pharmaceutical containing the controlled substance. For example, a teenager may be injured while participating in athletics and may, appropriately, be prescribed an opioid to manage pain associated with the injury. When the first prescription is consumed or during a follow up appointment, the teenager may legitimately (or illegitimately) request additional medication. Or, the care provider may perceive the teenager is still in pain and offer a refill of the prescription, a similar pharmaceutical, or a change in dosage. Or, the teenager's guardian may perceive the teenager is still in pain and request the same. In any case, during recovery the teenager can gradually develop a dependence on the pharmaceutical (e.g., the opioid in this case) and at some point addiction sets in. The teenager's life is detrimentally altered and the youthful potential possibly destroyed. Part of the challenge presented with addicting medications that are included in the controlled substances category is that the completion of the prescribed medications and the return of unused medication is not adequately tracked to ensure that unused prescriptions are returned, thereby limiting access to such medications and thereby decreasing the chances of misusing the medications.

Part of the addiction crisis has arisen due to the ease of acquiring controlled substances for non-medical purposes. While there are several circumstances in which prescribing controlled substances, such as an opioid, is medically appropriate, the inclination of medical professionals to prescribe controlled substances and the lack of adequate monitoring of prescribed medications results in thousands of unused medications remaining in the hands of individuals, which leads to unauthorized use of the controlled prescriptions by the individual prescribed or other individuals who may have access to the unused medications. For example, an individual may be prescribed oxytocin, a narcotic and controlled substance after undergoing wisdom tooth removal by an oral surgeon. However, it is not unusual, for an individual to not finish the prescription (e.g. not use all the pills prescribed) as the pain level experienced may be short lived or not high enough to require the stronger painkillers. When this happens, individuals are left with unused medications, which then may remain in prescription bottles within the individuals household or possession. This leads to potential opportunities for misuse of the medications—either by the individual or by someone else who may have access to the individual's medications. Unfortunately, individuals are not generally equipped with the information they need to properly dispose of unused medications and lack awareness of how and whereto dispose of unused medications. As the healthcare industry and society wrestle with the challenge of how to combat this crisis, one way to decrease the availability of controlled substances for unauthorized use is to provide methods for better individual awareness and disposal of unused prescriptions of controlled substances like opioids.

The opioid crisis has become so serious, that, as of 2018, there were about 192 people dying each day from opioid overdoses in the U.S. Opioid drugs are responsible for a majority of overdose deaths in the U.S. today. Each year there are over 650,000 opioid prescriptions dispensed daily and many of these medications wind up being misused for non-medical purposes. Often times, individuals are prescribed medications, such as opioids for valid medical reasons, but do not end up using or needing the full prescription. However, once done with the medication, individuals are rarely provided with the information as to what to do with left over, unused medications. Therefore, individuals either do not dispose of the medications or may dispose improperly, thereby providing opportunities for potential misuse by the prescribed individual or others.

In fact, according to studies, almost 60% of Americans have opioid painkillers stored at home that are no longer in use. Studies have shown that 65% of prescription pain relievers for non-medical use in previous years have been the result of receiving from a friend/relative for free, bought from a friend or relative, or taken from a friend or relative without asking. These statistics emphasize the importance of a system that would provide individuals with the with the proper information to then return unused medications, thereby decreasing the chances that prescribed individuals will utilize prescription medications for non-medical use or other individuals will get their hands on the medications. It is critical to increase awareness to combat the issue so that unused medications are safely disposed of, limiting the availability of unused opioids that contribute to the current crisis. This can be done by connecting data points in healthcare systems and utilizing the electronic medical records with other data to pinpoint the prescriptions prescribed, the directed length of use for the prescription, and providing individuals with the location of collection centers located near the individuals for safe disposal of any unused medications. Accordingly, the embodiments described herein, at least partially, prevent situations like those mentioned above from occurring through improvements to current electronic health record (EHR) system technology. For example, systems and methods described herein dynamically engages with individuals to increase the return of unused controlled substance prescriptions to one or more identified controlled centers. This will decrease the likelihood that unused prescription medications will wind up being used for non-medical purposes, thereby decreasing the likelihood individuals will have easy access to prescription medications for non-medical reasons and positively impact the current addiction crisis by decreasing access to controlled substances and thereby impacting the addiction rates.

Embodiments of the present invention generally relate to computerized systems and methods for dynamically engaging with individuals to increase return of unused controlled substance prescriptions to one or more collection centers. The methods and system utilize one or more indications received that an individual has been prescribed a first medication and data received that is associated with the first medication. Based on this information, the system determines whether the first medication is a controlled substance. Upon determining that the first medication is a controlled substance, a first set of data associated with an individual and a second set of data associated with one or more collection centers are identified. Then, the systems and methods determine one or more collection centers located within a predetermined distance from the individual based on the first set of data associated with the individual and the second set of data associated with one or more collection centers. The systems and methods further identify a third set of data associated with the prescribed medication and generate a report for the individual comprising one or more of the first set of data associated with the individual, the second set of data associated with the one or more collection centers, the third set of data associated with the prescribed medication, and the determined one or more collection centers. The systems and methods further determine a stop date for the medication and transmit a message to the individual on a user interface reminding the individual of the determined stop date of the first medication and the one or more collection center locations for return of any unused first medication.

In additional embodiments, the present invention generally relates to methods for dynamically engaging with a plurality of individuals to increase return of unused controlled substance prescriptions to one or more collection centers. One or more or more indications that a plurality of individuals have been prescribed a first medication are received. Additionally, data associated with the first medication is received. Then, whether the first medication is a controlled substance is determined. In response to determining that the first type of medication is a controlled substance, a first set of data associated with each of the plurality of individuals prescribed the first medication is identified and a second set of data associated with one or more collection centers is identified to determine one or more collection centers located within a predetermined distance from each individual of the plurality of individuals. The method further identifies a third set of data associated with the prescribed medication and then generates a report for each individual of the plurality of individuals comprising one or more of the first set of data associated with each individual, the second set of data associated with the one or more collection centers, the third set of data associated with the prescribed medication, and the determined one or more collection centers. Additionally, the method further determines a stop date for the first medication and transmits a message to each individual of the plurality of individuals on a user interface reminding each individual of the determined stop date of the first medication and the one or more collection center locations for return of any unused first medication.

### BRIEF DESCRIPTION OF THE DRAWINGS

The present invention is described in detail below with reference to the attached drawing figures, wherein:

FIG. 1 depicts an exemplary operating environment consistent with embodiments described herein;

FIG. 2 is an exemplary system architecture suitable to implement embodiments of the present invention, in accordance with aspects herein;

FIG. 3 is illustrates an exemplary process of how an exemplary system utilizes various data to provide an individual with information to return unused controlled substance prescribed, in accordance with aspects herein;

FIG. 4 illustrates another exemplary process illustrating the process of dynamically engaging with an individual at a point of care by providing collection center information to increase the return of unused prescription medications;

FIG. 5 illustrates yet another exemplary process of dynamically engaging with individuals at a point of care by providing collection center information to the individual via text message to increase the return of unused prescription medications;

FIG. 6 illustrates an exemplary method for dynamically engaging with an individual to increase return of unused controlled substance prescriptions to one or more collection centers, in accordance with aspects herein;

FIG. 7 illustrates an exemplary method for dynamically engaging with a plurality of individuals to increase return of unused controlled substance prescriptions to one or more collection centers; and

FIG. 8 illustrates an exemplary chart depicting how the method of FIG. 17 engages with a plurality of individuals to determine whether the medication prescribed is a controlled substance, the collection centers for each of the individual, and the order stop dates for each individual.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

A system, as used herein, refers to any device, process, or service or combination thereof. As used herein, engine is synonymous with system unless otherwise stated. A system may be implemented using components or generators as hardware, software, firmware, a special-purpose device, or any combination thereof. A system may be integrated into a single device or it may be distributed over multiple devices. The various components or generators of a system may be co-located or distributed. For example, although discussed for clarity as the prediction engine, operations discussed may be performed in a distributed manner. The system may be formed from other systems and components thereof. It should be understood that this and other arrangements described herein are set forth only as examples.

As used herein, an electronic medical record (EMR) or electronic health record (EHR) maintains, and provides access to, one or more EHR database(s) containing records of treatment events, medication history, substance use history, demographic attributes, laboratory tests, time and date data, any other health related data, or any combination thereof for a plurality of patients. For example, an EHR can include THC metabolites, opioids, opioid metabolites (e.g., the metabolites of heroin, opium, morphine, hydrocodone, oxycodone, and so forth), and other pharmaceuticals or metabolites thereof. Additionally, the EHR can include clinical notes, appointment notes, records of issued prescriptions, diagnoses, care plans, bloodwork, urinalysis, respiratory data, and treatment data for each patient of a healthcare institution or a plurality of healthcare institutions. Further, an EHR can include images, representations, or clinical documentation of burns, needle marks or unexplained abrasions, shakes/tremors, lack of personal hygiene, scars, unsubstantiated pains, requests for opioids or pain medication, etc. Additionally, in some embodiments, the EHR can maintain one or more pharmaceutical formularies that identify the chemical composition of pharmaceuticals prescribed by, or available for prescription by, care providers. The formularies can further include keywords, potential for addiction information, dosage guidelines, generic and non-generic names, national drug codes (NDC), and any other data related to prescribeable pharmaceuticals. In some embodiments, a specific potential-for-addiction formulary can include a subset of pharmaceuticals with a known potential for abuse or addiction.

As described herein, a controlled substance is a drug or chemical whose manufacture, possession, or use is regulated by the government such as illicitly used drugs or prescription medications. These medications can have detrimental effects on a person's health and welfare, which is why they are regulated by the federal and state governments. The federal government has defined a controlled substance as any substance listed in the schedules of the Control Substances Act of 1970 and breaks the schedules down into five categories. Schedule 1 includes substances that have no acceptable medical use, are unsafe and hold a high potential for abuse. These include heroin, LSD, marijuana, and ecstasy. Schedule II include narcotics and stimulants that have a high potential for abuse and engender severe psychological or physical dependence. Examples include Dilaudid, hydrocodone, methadone, Demerol, OxyContin, Percocet, morphine, opium, codeine, amphetamine (Dexedrine, Adderall), and methamphetamine. Schedule III includes substances that have less potential for abuse but can still lead to moderate or low physical dependence and high psychological dependence. They include Tylenol with Codeine, Suboxone, ketamine, and anabolic steroids. Schedule IV substances have a lower potential for abuse than Schedule III drugs, and include Xanax, Soma, Klonopin, Valium, Ativan, Versed, Restoril, and Halcion. Finally, Schedule V substances are primarily preparations that contain limited quantities of narcotics, including cough syrups that contain codeine. While it is technically illegal to possess any drug listed in the categories above, an individual has not violated the law if any substance is properly prescribed to an individual. The current opioid and narcotic addiction crisis has resulted from the misuse of medications that primarily fall within Schedule II and III.

Pharmacological advancements have created numerous beneficial medications that can relieve pain, combat depression, lower blood pressure, or balance neurochemistry amongst a plethora of other physiologic and psychologic benefits. However, addiction to medications (such as opioids, barbiturates, benzodiazepines, phenidates, amphetamines, and other controlled substances) can be as destructive to a person as the medication's therapeutic use can be beneficial. While the most destructive consequence of addiction may be the death of the person or someone else, other common effects of addiction can include impaired behavioral control, craving, diminished recognition of significant problems with one's behaviors and interpersonal relationships, and dysfunctional emotional responses, among other things.

On an individual basis, the effects of addiction are tragic. On a societal level the expenditure of healthcare resources treating addiction and relapse of an increasingly large number of people can alter the availability and quality of care for everyone. Unfortunately, the gradual deterioration of a patient from legitimate therapeutic use through initial dependence to addiction creates unique prediction, detection, and prevention problems that may not be addressed by the traditional EHR systems used in the healthcare system. Further, the proliferation of urgent care, surgi-centers, rehabilitation clinics, satellite care centers, and other public or private clinics means that patients often interact with multiple care providers across multiple institutions. These institutions commonly structure the data within their respective EHR systems differently from other institutions (e.g., the organization of a database table, the subsystem that maintains a record(s), the programmatic language, and so forth), use different versions of the EHR system at different locations (e.g., a community clinic using a “lite” or older version while the level 3 trauma center in the heart of the city uses the newest iteration), or have different data formatting procedures (e.g., including/omitting middle initials, month/day/year or year/month/day, including/omitting a unit identifier, metric/imperial). These differences mean that identifying and collecting patient information across multiple disparate traditional EHR systems can be prevented based on incompatibilities between the sources of patient data.

The current healthcare crisis surrounding opioid and other prescription medication overdose and abuse is well-known and a critical problem in the healthcare field. In fact, according to studies, 6 million Americans misused controlled prescription drugs and a majority of the misused prescription drugs were obtained from family members and friends, often from the home medicine cabinet. In some circumstances, friends or family members will give away, allow, or even sell old prescription medications or controlled substances without realizing the potential risk of addiction and overdose. In other circumstances, individuals may be aware of unused controlled substances in a family member of friend's possession and may take them without permission. Regardless of how the controlled substances are obtained, our society is facing a crisis managing addiction and overdose caused by controlled substance abuse. In many circumstances, individuals do not have the intent to abuse controlled substances prescribed to them and often do not dispose of such medication as a result of lacking information regarding how to properly dispose of unused medication. In light of the complex circumstances, the healthcare industry needs a method for utilizing technology to connect data regarding individuals prescribed controlled substances with other reference data available in an electronic medical record or healthcare system in order to provide individuals with information on where and how to properly dispose of unused controlled substances.

As such, the present disclosure discusses systems and methods for dynamically engaging with individuals to increase return of unused controlled substance prescriptions to one or more collection centers. Further, determinations and reminders for individuals that the time period for use of a controlled substance prescription has expired or when it will expire may increase the likelihood that individuals will take the time to drop off unused controlled substance prescriptions to authorized locations for safe removal of the unused medications. Additionally, a method that provides individuals with one or more collection centers for drop off of the unused controlled substances that are within a predetermined distance from the individuals location will make it even easier for individuals to drop off unused medications and further increase the likelihood that such medications will be returned instead of remaining in household medicine cabinets for months or years providing the opportunity for misuse. While the current prescription medication abuse and overdose crisis will require several different solution to solve, dynamically engaging with individuals to provide them with the information regarding the location of drug collection centers, timeframe for when prescription end dates occur, and reminders to return unused medications to a collection center will have a positive impact in decreasing the number of medication accessed for non-medical purposes through friends and family and will further create better patient awareness which is key to ending the current crisis.

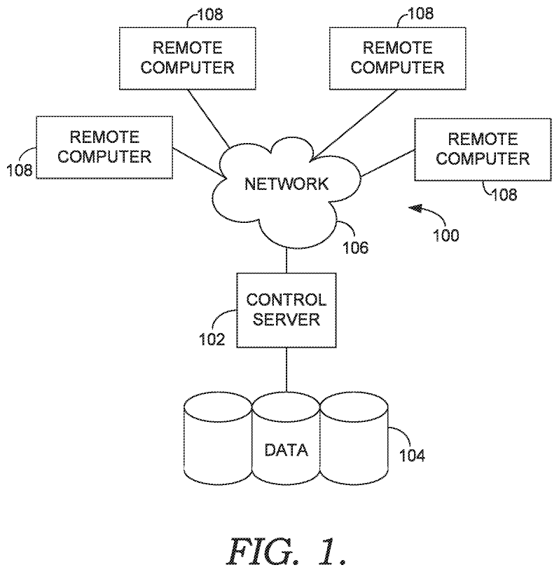

Beginning with FIG. 1, an exemplary computing environment suitable for use in implementing embodiments of the present invention is shown. FIG. 1 is an exemplary computing environment (e.g., health-information computing-system environment) with which embodiments of the present invention may be implemented. The computing environment is illustrated and designated generally as reference numeral 100. The computing environment 100 is merely an example of one suitable computing environment and is not intended to suggest any limitation as to the scope of use or functionality of the invention. Neither should the computing environment 100 be interpreted as having any dependency or requirement relating to any single component or combination of components illustrated therein. It will be appreciated by those having ordinary skill in the art that the connections illustrated in FIG. 1 are also exemplary as other methods, hardware, software, and devices for establishing a communications link between the components, devices, systems, and entities, as shown in FIG. 1, may be utilized in the implementation of the present invention. Although the connections are depicted using one or more solid lines, it will be understood by those having ordinary skill in the art that the exemplary connections of FIG. 1 may be hardwired or wireless, and may use intermediary components that have been omitted or not included in FIG. 1 for simplicity's sake. As such, the absence of components from FIG. 1 should not be interpreted as limiting the present invention to exclude additional components and combination(s) of components. Moreover, though devices and components are represented in FIG. 1 as singular devices and components, it will be appreciated that some embodiments may include a plurality of the devices and components such that FIG. 1 should not be considered as limiting the number of a device or component.

The present technology might be operational with numerous other special-purpose computing system environments or configurations. Examples of well-known computing systems, environments, and/or configurations that might be suitable for use with the present invention include personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be operational and/or implemented across computing system environments such as a distributed or wireless “cloud” system. Cloud-based computing systems include a model of networked enterprise storage where data is stored in virtualized storage pools. The cloud-based networked enterprise storage may be public, private, or hosted by a third party, in embodiments. In some embodiments, computer programs or software (e.g., applications) are stored in the cloud and executed in the cloud. Generally, computing devices may access the cloud over a wireless network and any information stored in the cloud or computer programs run from the cloud. Accordingly, a cloud-based computing system may be distributed across multiple physical locations.

The present technology might be described in the context of computer-executable instructions, such as program modules, being executed by a computer. Exemplary program modules comprise routines, programs, objects, components, and data structures that perform particular tasks or implement particular abstract data types. The present invention might be practiced in distributed computing environments where tasks are performed by remote processing devices that are linked through a communications network. In a distributed computing environment, program modules might be located in association with local and/or remote computer storage media (e.g., memory storage devices).

With continued reference to FIG. 1, the computing environment 100 comprises a computing device in the form of a control server 102. Exemplary components of the control server 102 comprise a processing unit, internal system memory, and a suitable system bus for coupling various system components, including data store 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 102 typically includes therein, or has access to, a variety of non-transitory computer-readable media. Computer-readable media can be any available media that might be accessed by control server 102, and includes volatile and nonvolatile media, as well as, removable and nonremovable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by control server 102. Computer-readable media does not include signals per se.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

The control server 102 might operate in a computer network 106 using logical connections to one or more remote computers 108. Remote computers 108 might be located at a variety of locations including operating systems, device drivers and the like. The remote computers might also be physically located in traditional and nontraditional clinical environments so that the entire medical community might be capable of integration on the network. The remote computers might be personal computers, servers, routers, network PCs, peer devices, other common network nodes, or the like and might comprise some or all of the elements described above in relation to the control server. The devices can be personal digital assistants or other like devices. Further, remote computers may be located in a variety of locations including in a medical or research environment, including clinical laboratories (e.g., molecular diagnostic laboratories), hospitals and other individual settings, veterinary environments, ambulatory settings, medical billing and financial offices, hospital administration settings, home medical environments, and clinicians' offices. Medical providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like. The remote computers 108 might also be physically located in nontraditional clinical environments so that the entire medical community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networking environment, program modules or portions thereof might be stored in association with the control server 102, the data store 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a microphone (e.g., voice inputs), a touch screen, a pointing device (commonly referred to as a mouse), a trackball, or a touch pad. Other input devices comprise satellite dishes, scanners, or the like. Commands and information might also be sent directly from a remote medical device to the control server 102. In addition to a monitor, the control server 102 and/or remote computers 108 might comprise other peripheral output devices, such as speakers and a printer.

Although many other internal components of the control server 102 and the remote computers 108 are not shown, such components and their interconnection are well known. Accordingly, additional details concerning the internal construction of the control server 102 and the remote computers 108 are not further disclosed herein.

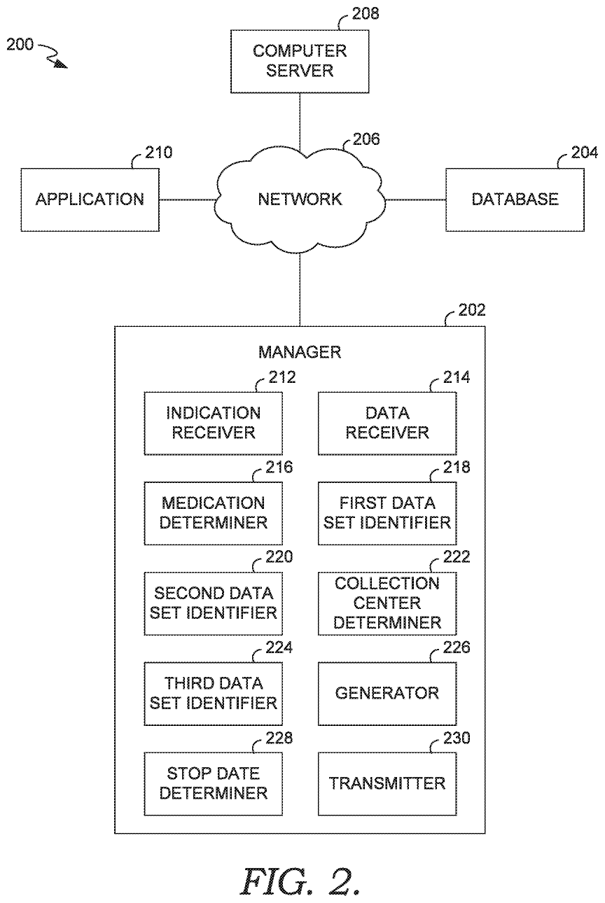

Turning now to FIG. 2, a system 200 for dynamically engaging with an individual to increase return of unused controlled substance prescriptions to one or more collection centers is depicted. The exemplary system 200 is merely an example of one suitable system and is not intended to suggest any limitation as to the scope of use or functionality of embodiments of the present invention. Neither should the system 200 be interpreted as having any dependency or requirement related to any single component or combination of components illustrated herein.

The exemplary system 200 comprises a manager 202, a database 204, a network 206, a computer server 208, and an application 210. As shown, FIG. 2 includes one manager 202, one database 204, one network 208, and one application 210. However, it is contemplated that the system 200 may comprise more than one of each of these components depending on the needs of the system 200. For example, the system 200 may comprise more than one database 204, which may be located remotely or on the cloud.

As depicted, the system 200 comprises manager 202. It will be appreciated that some or all of the subcomponents of the manager 202 may be accessed via the network 206 and may reside on one or more devices. Additionally, the manager 202 may also be integrated into the application 210. Further, in some embodiments, one or more of the illustrated components may be implemented as a stand-alone application. The components described are exemplary in nature and in number and should not be construed as limiting. Any number of components may be employed to achieve the desired functionality within the scope of the embodiments hereof.

Generally, the manager 202 is configured to help increase the return of a unused medication determined to be a controlled substance by generating a report for an individual that comprises information such as the location of one or more collection centers within a predetermined distance from the individual. The manager 202 is further configured to generally determine a stop date for the first medication and transmit a message to the individual prescribed the first medication comprising the stop date of the first medication and collection center locations for return of any unused first medications. As shown, the manager 202 is comprised of several components including: an indication receiver 212, a data receiver 214, a medication determiner 216, a first data set identifier 218, a second data set identifier 220, a collection center determiner 222, a third data set identifier 224, a generator 226, a stop date determiner 228, and a transmitter 230. In embodiments, the manager 202 can include any number of components necessary for building at least one custom documentation template.

The indication receiver 212 within manager 202 is configured to receive one or more indications that an individual has been prescribed a first medication. Such indications may occur when a user, such as a healthcare provider, inputs certain information into an application (such as application 210) or electronic health record system. Exemplary one or more indications may include input of medication orders during a hospital visit or discharge summary, during an outpatient encounter with an individual or when medications are either automatically refilled or refilled upon request at a pharmacy. For example, if an individual has a scheduled encounter with a pain management physician regarding continued pelvic pain due to an injury from a recent car accident, the individual may be prescribed a controlled substance, such as the painkiller oxytocin, by the pain management physician in order to help manage the individual's constant pain. At the time the pain management physician inputs the medication order or submits an electronic prescription for the oxytocin, the system will receive an indication that the individual has been prescribed a first medication.

In other circumstances, the system may receive the indication based on a medication refill sent to a pharmacy from a physician. In this case, the system may receive the indication that the refill for the controlled substance has been initiated or that the individual has picked up the prescription for use. When this occurs, the indication receiver 212 may receive the indication that the first medication has been prescribed to the individual via an EHR or communications via the network with a computer server or application connected with a pharmacy. Further, the indication receiver 212 may also receive the indication that a first medication has been prescribed for the individual upon discharge of the individual from the hospital. For example, if an individual breaks their hip and is hospitalized and undergoes surgery, at the time of discharge, the system 200 may receive, via the indication receiver 212, an indication that the individual was prescribed a first medication (a controlled substance such as oxytocin) for pain management.

Once the system 200 has received the one or more indications that the individual has been prescribed a first medication, the data receiver 214 will receive data associated with the first medication. The data associated with the first medication received by the data receiver 214 may include, but is not limited to, an encounter ID, dosage of medication and prescribed intake, medical provider license number, name of the medication, date of prescription, side effects, and any additional relevant information regarding the first medication.

Once the data associated with the first medication is received by the data receiver 214, a medication determiner 216 will determine whether or not the first medication prescribed is a controlled substance. The medication determiner 216 may determine this information by checking with a drug database which includes information regarding prescription medications and their class or schedule categorizations described above. The database utilized may be the system's own database, such as one built within an EHR system, may be a publicly available, or may be a private database separate from the EHR system. In some aspects, the medication determiner 216 may determine that the medication prescribed is not a controlled substance. When this occurs, no further action is needed, as the first medication is not a controlled substance and therefore not a medication considered to pose a significant threat if unused and unreturned. In aspects discussed in the present disclosure, the medication determiner 216 will reference a drug database and determine that the first medication prescribed is a controlled substance.

Upon the determination that the first medication prescribed is a controlled substance by the medication determiner 216, a first data set identifier 218 will identify a first data set associated with the individual. The first data set identifier 218 may identify the first data set from a database 204, a remote computer server 208, or an application such as application 210. It is contemplated that the first data set identifier 218 may identify the first data set from any resource available that comprises data associated with the individual. The first set of data associated with the individual includes, but is not limited to, the individual's zip code, address, mobile phone number, insurance number, and other biographical data that is relevant in the process. For example, the first set of data identified may comprise the zip code of the individual, the address of the individual, the insurance information, including whether or not the individual is the primary enrollee or subscriber for the insurance. In circumstances where the individual being prescribed the first medication is not the primary subscriber, additional data such as the primary subscriber's date of birth, social security number, insurance member and group ID, and mobile phone number may also be included in the first set of data identified.

Additionally, a second set of data is identified by a second data set identifier 220. The second set of data identified by the second data set identifier 220 comprises data associated with one or more collection centers. As defined herein, collection centers are physical locations designated by either the Drug Enforcement Agency (DEA), another government agency, a healthcare facility or organization, or any other authorized party for collection of unused medications that are controlled substances and fall within certain class schedules associated with high risk. The DEA has designated controlled substance public disposal locations around the country and provides for searching for the nearest location on their website. When the second data set identifier 220 identifies the second set of data, it will identify collection center details such as the name and address of the collection center. This information will be determined by utilizing the zip code from the first set of data identified by the first data set identifier 218. Additional information regarding the collection center may also be identified by the second data set identifier 220 such as the hours of operation for the collection center, which types of controlled substances are accepted at each collection center, and any other requirements for disposal.

Once the second data set identifier 220 identifier the second data set, the collection center determiner 222 will determine or more collection centers located within a predetermine distance from the individual. Using the zip code of the individual, the collection center determiner 222 will identify at least one collection center within a predetermined distance from the individual. For example, in some aspects, the collection center determiner may identify three or more collection centers that are within a 10 mile radius of the individual. In other circumstances, the collection center determiner 222 may identify multiple collection centers that are within a smaller or larger distance from the individual, depending on the individual's location and the availability of collection centers within the predetermined distance of the individual. The collection center determiner 222 will determine the one or more collection centers based on both the first set of data associated with the individual identified by the first data set identifier 218 and the second set of data associated with the one or more collection centers identified by the second data set identifier 220. The system 200 will analyze the first set of data and the second set of data and utilize smart address text mapping with individual and collection center.

Once the collection center determiner 222 determines the one or more collection centers, a third data set identifier 224 will identify a third data set that is associated with the prescribed medication. The third data set associated with the prescribed medication includes, but is not limited to, an encounter ID, an order or prescription start date, and an order or prescription stop date. The third data set identifier 224 may identify the third set of data from a database, like database 204, an application such as application 210, or a local or remote computer server such as computer server 208. The third set of data associated with the first prescribed medication may be from any type of encounter, such as an outpatient physician visit, an emergency room or hospital encounter and subsequent discharge, or a refill of a medication. Each type of encounter may have an encounter ID associated with the encounter and the ID may be set of numbers, letters, or any designation method for identifying the encounter from which the first medication was prescribed. For example, if individual A was prescribed a controlled substance, such as hydromorphone, for pain management related to a slipped disc in the back, A's encounter with the physician in which she was prescribed the hydromorphone would include an encounter ID (e.g. SPF12345).

Additionally, the third set of data would include a start date and an end date for the prescribed hydromorphone. Continuing with this example, if individual A just underwent surgery for her slipped back disc and is being discharged from the hospital, the discharging physician may prescribe her 24 pills of hydromorphone with orders to take one pill every 6 hours starting with day after discharge as needed. As such, if individual A is discharged on June 30, her prescription will run out, in 6 days or on July 6 if she begins taking the hydromorphone on July 1. Therefore, her order start date is July 1 and the order stop date is July 6. However, individual A may not need all the hydromorphone prescribed and may decide on the third day to only take one hydromorphone pill each day for the next three days. As such, on July 6 (day 6), individual A would have only utilized 11 hydrophone pills, resulting in 13 unused pills that should be identified for return to a collection center determined by the collection center determiner 222.

Additionally, a generator 226 generates a report for the individual (such as individual A) comprising one or more of the first set of data associated with the individual, the second set of data associated with the one or more collection centers, the third set of data associated with the prescribed medication, and the determined one or more collection centers. It is contemplated that the report generated may include all three sets of data, only one set of the three sets of data, or portions of each set of data depending on what is needed to help increase the likelihood that the individual will return any unused medications to a collection center. Additionally, the generator 226 may generate the report during different points of care such as when an outpatient prescription is picked up at a pharmacy or clinic, when a physician inputs the prescription either during a scheduled physician visit or during an unplanned encounter (e.g. emergency room visit). Further, the generator 226 may generate the report for the individual as a portion of an inpatient discharge summary. In additional aspects, the generator 226 may generate the report of the individual upon the ordering and/or pickup of a medication refill. Moreover, it is contemplated that an individual retrieving a medical refill may have more than one report generated by the generator 226. For example, when an initial prescription is ordered during an outpatient encounter with a physician, the generator 226 may generate a report related to the outpatient prescription. Then, a second report, may be generated by the generator 226 upon the medication being refilled. In these circumstances, the reports may differ in the start and end date of the prescription, the number of pills prescribed, and the dosage. Additionally, when the generator 226 includes the first set of data associated with the individual in the report, the report may comprise all the data identified that is associated with the individual including their address, mobile number, zip code, and other biographical information. When the generator 226 includes the second set of data in the report generated, the report will include information regarding the one or more collection centers including the collection centers locations, names, addresses, distance from the individual to each collection center, and other relevant information.

Once the report has been generated for the individual by the generator 226, a stop date determiner 228 will determine the stop date for the first medication. In some other aspects, the stop date determiner 228 may determine the stop date prior to the generation of the report by the generator 226. As mentioned, the stop date may be based on the number of pills prescribed and their directed dosage/day. For example, a physician may prescribe 20 oxytocin pills to be taken once a day starting on July 1^{st}. Therefore, if taken once a day, the prescription will end or stop on July 20^{th}. This means that, if taken properly, there will be no additional medication available for the prescribed reasons (e.g. pain management) after twenty days. However, in some circumstances, an individual may decide not to take the oxytocin for more than 5 days. If that is the case, the stop date will remain July 20, but there will be remaining medication that is unused and should be proposed disposed of by returning to a collection center.

Once the stop date for the first medication is determined by the stop date determiner 228, a transmitter 230 transmits a message to the individual on a user interface reminding the individual of the determined stop date for the first medication and the one or more collection centers of any, a transmitter 230 will transmit a message to the individual informing them of the stop date of the first medication and the one or more collection center locations for return of any unused medication. It is contemplated that the message transmitted may be a text message, email, or any other electronic communication that the individual is capable of receiving. Additionally, in some aspects, the transmitter 230 will send a second message to the individual after the stop date has past to remind the individual to dispose of the unused medication at one or more of the collection centers identified.

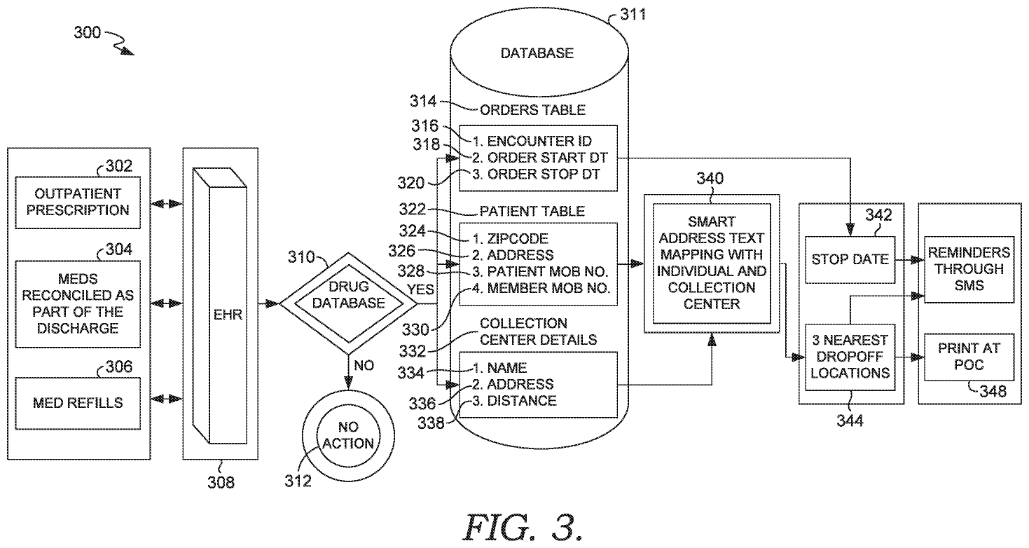

Next, FIG. 3. illustrates an exemplary process 300 of providing individuals with information to return unused controlled substance prescribes, in accordance with aspects herein. As shown, the process 300 begins with one of three points of care occurring: an outpatient prescription 302, a medication reconciliation as part of a patient discharge from a hospital 304, and medication refills 306. When any of these three points of care occur and result in a prescription of a first medication, the indication receiver 212 will receive an indication that a first medication has been prescribed for an individual. As mentioned, the indication may occur as a result of input into an EHR such as EHR 308 or as a result of EHR 308 receiving indications that one of the three points of care have occurred. Once the EHR 308 has an indication, either via direct input into the EHR or some other indication, the EHR 308 communicates with drug database 310. Drug database 310 may be a publicly available drug database or an application that the EHR 308 may communicate with. The EHR 308 communicates with the drug database to determine whether or not the first medication prescribed is a controlled substance. If the first medication is not one that falls within the controlled substance list in the drug database 310, then no further action is needed.

If the first medication prescribed falls within the controlled substance category (e.g. Schedule II or III), then the process 300 continues. The system will reference three tables within a database 311 in order to identify multiple sets of data. The first set of data identified by the first data set identifier 218 will be data associated with the individual seen in the patient table 322. As shown, the patient table 322 includes data such as the zip code 324, address 326, patient mobile number (patient mob. no.) 328 and member mobile number (member mob. no.) 330. As previously mentioned, the first set of data may include additional information associated with the individual such as the individual's social security number or date of birth. Additionally, the system 200 will also identify a second set of data, via the second data set identifier 220, that is associated with one or more collection centers. As shown in FIG. 3, the second set of data comprises the collection center details 332 which include, but are not limited to the name 334, address 336, and distance 338 of the one or more collection centers.

Once the patient table 322 and collection center details 332 are utilized to identify the first set of data associated with the individual and the second set of data associated with one or more collection centers, the process 300 then utilizes smart address text mapping to determine one or more collection centers that are located within a predetermined distance from the individual at 340. Additionally, as shown, the process 300, utilizing the smart address text mapping, may determine the three nearest drop off locations 344 for unused medications. By determining the three nearest drop-off locations based on the first set of data related to the patient and the second set of data comprising collection center details, the exemplary process 300 provides an individual with information regarding convenient locations for dropping off unused medication.

Additionally, once the nearest drop off locations are determined at 344, the information is generated by generator 226 in a report that is then printed at the point of care. In other words, the individual may receive the information for the nearest collection center to the individual's location at the time the individual receives the first medication (e.g. discharge 304, outpatient prescription 302, and medication refills 306). By providing this information at the time the patient receives the medication, the process 300 makes the process of returning unused medications even easier on the individual. The individual does not need to follow up, do research, or any additional work aside from actually dropping off any unused medication after the prescription stop date to the collection center. This may lead to an increase in returned unused controlled substances as the reality is that most individuals do not return unused prescription medications, especially those that are controlled, out of any intention to misuse the medications, but out of either laziness or inconvenience of the process. As such, by providing making the information for where drop off collection centers are located within a close distance to the individual at the point of care, it increases the likelihood that when prescriptions are unused, an individual will drop them off at a collection center. By encouraging this and making the information readily available, this will lead to a decrease in the availability of unused prescription medications, which thereby may help decrease the instances where individuals are either misusing prescription medications prescribed to them for medical purposes or are providing, unintentionally or intentionally, unused prescription medications to other individuals for non-medical purposes.

Additionally, the process 300 identifies a third set of data that is associated with the prescribed first medication, via the third data set identifier 224. As shown in FIG. 3, an orders table 314 is accessed in the database 311 to identify the third set of data. The third set of data associated with the prescribed medication includes, but is not limited to, an encounter ID 316, order start date 318 and an order stop date 320, which have been described previously. Based on the information identified in the orders table 314, a stop date for the first medication will be determined at 342. Once the stop date 342 has been determined, one or more reminders or messages 346 may be sent to the individual, family members, guardians, and/or caretakers on a user interface reminding the individual of the stop date of the first medication and the one or more collection centers for return of any unused first medication. In some instances, an individual may receive one message, such as an SMS text message, that includes both the nearest collection centers for drop off and the stop date of the first prescription. In other instances, the information may be transmitted via more than one message to the individual. Additionally, the process may follow up with additional reminders just prior to the stop date, on the stop date, and after the stop date in an effort to remind the individual that the time period for using the medication as prescribed has ended and therefore any unused medication should be properly disposed of at one of collection centers identified.

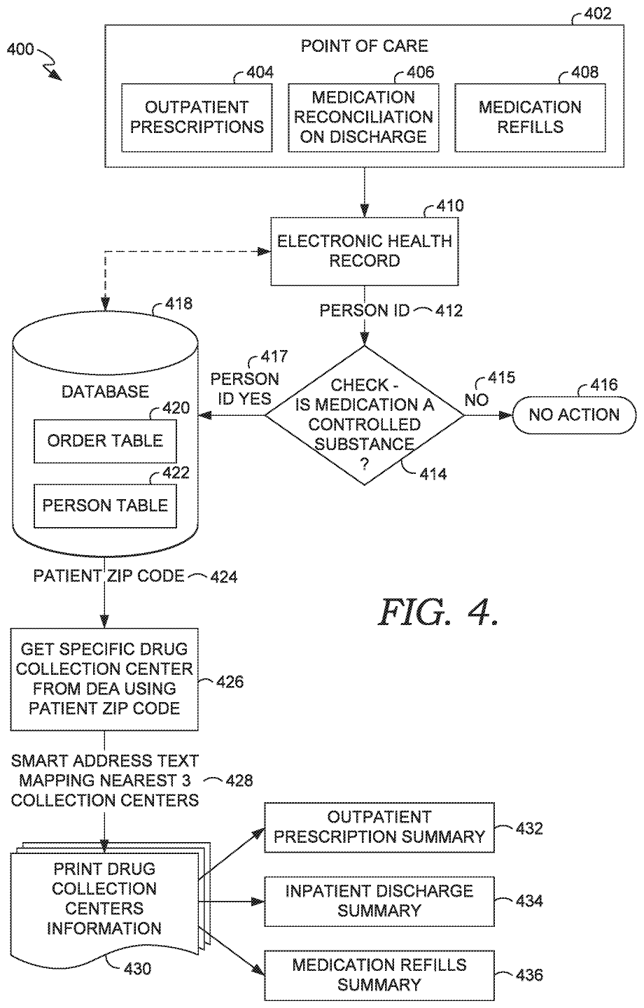

Continuing on, FIG. 4 illustrates another exemplary process illustrating the process of dynamically engaging with individuals at a point of care by providing collection center information to increase the return of unused prescription medications. Exemplary process 400 begins with the point of care 402. As shown and previously described, the point of care 402 may include when an outpatient prescription 404 is input or ordered by a healthcare provider, when medication reconciliation on discharge 406 occurs, or when medication refills 408 are completed. While these three exemplary point of cares are discussed, it is contemplated that other points of care that may result in prescription medication orders may also be included in the point of care 402 and the point of care 402 illustrated is not limited to only the three events shown. The occurrence of a point of care 402 is either reported to the EHR 410 or the directly input into the EHR 410 by either the healthcare provider or some other authorized individual. Then, data associated with the individual is identified by identifying the individual or person identification 412.

Following this, whether or not the medication is a controlled substance is determined. Utilizing a drug database, the system 200, will determine whether the first medication is a controlled substance. This may occur by the manual input of the medication into an on-line database system or through the use of a drug database system present within the EHR. If the answer to the question of whether or not the medication is a controlled substance 414 is a “no” as seen at 415, then the process ends and no further action 416 is needed.

However if the process 400 determines that the medication prescribed is a controlled substance, then the process continues with identifying a first set of data associated with the individual and a third set of data associated with the prescribed medication from database 418. In this case, the first set of data in database 418 is the personal table 422, which includes at least the identification of the zip code, addresses, patient mobile number, and the patient supporting member (family, guardian, etc.) member mobile number. Additionally, the third set of data associated with the first medication is identified via the orders table 420 in the database 418 and includes at least an encounter ID, order start date, and order stop date. Once the first set of data and the second set of data are identified, the patient zip code 424 is identified from the personal table 422. This information is then utilized to determine the specific drug collection center to be provided to the individual from the DEA at 426. The DEA has authorized various collection centers around the country, and the process will utilize personal information such as the location, to find one or more of the drug collection centers.

Once the collection centers are determined at 426, smart address text mapping 428 identifies the three collection centers closest to the individual. Then, the report is generated for the individual and includes the drug collection center information, including the location of the three nearest collection centers. The report generated by the generator 226 is printed for the individual at 430 during one of the points of care. It is contemplated that the individual may receive the report comprising the nearest drug collection centers on paper. The drug collection center information may be printed off on an outpatient prescription summary 432, an inpatient discharge summary 406, or medication refill summary 436. As such, the report generated is given to the individual prior to the end of the encounter and during one of the points of care 404. For example, prior to picking up the outpatient medication at the pharmacy, the outpatient prescriptions summary report that comprises the collection center information determined may be provided along with the prescribed medication. As such, in this example, a pharmacist may be provided with the report generated by the generator 226 and may review the report in additional to other prescription intake instructions with the individual at the time of prescription pick up. It is further contemplated that in other aspects, the report generated may be electronically communicated to the individual via email, text message, or any other suitable electronic communication means.

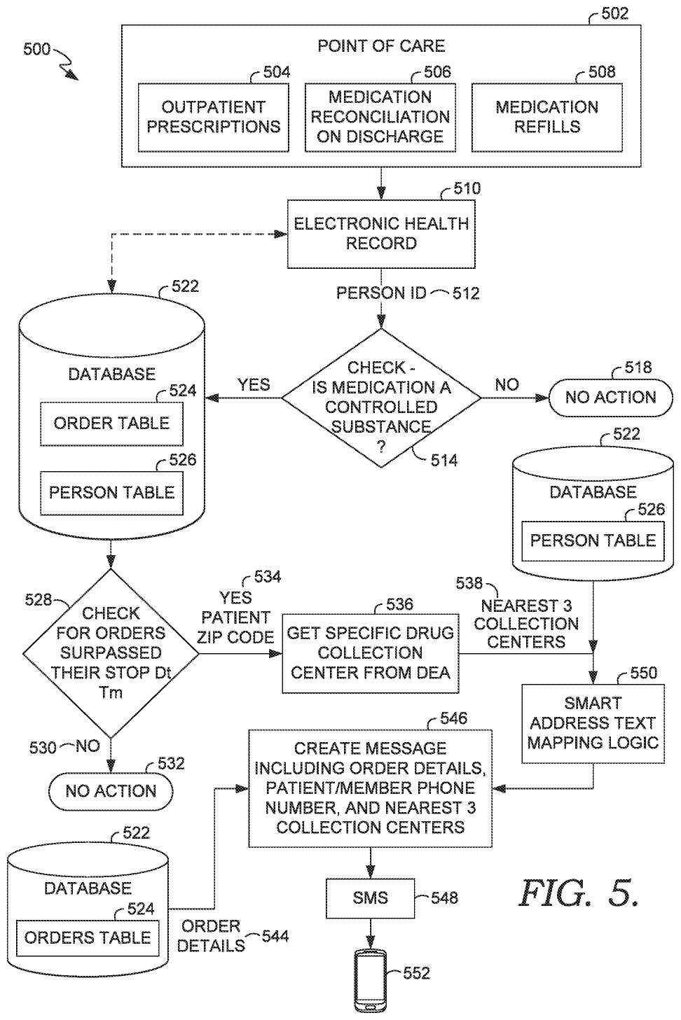

Next, FIG. 5 illustrates another exemplary process 500 of dynamically engaging with individuals at a point of care by providing collection center information to increase the return of unused prescription medications. Similar to the process 400 in FIG. 4, process 500 begins at a point of care 502. As shown, the exemplary points of care 502 trigger the process to begin when one or more indications are received that an individual has been prescribed a first medication. These indications may occur as a results of an outpatient prescription 504, a medication reconciliation on discharge from a hospital 506, or medication refill 508. While these three points of care are shown in FIG. 5, it is contemplated that other points of care may similarly trigger the process 500. Once the indication has been received that an individual has been prescribed a first medication by the EHR 510, then the identification of the personal or individual 512 is completed. Following this, the process 500 includes checking on whether the prescribed medication is a controlled substance at 514. If the medication is determined not the be a controlled substance, then no further action 518 is needed by the process and the individual may dispose of the unused medication in any manner.

If it is determined that the medication is a controlled substance, then the process 500 utilizes database 522 to obtain a first set of data associated with the individual by utilizing the data available in the person table 526. The process 500 further utilizes database 533 to identify a third set of data associated with the prescription via the data available from the orders table 524. As previously discussed the person table 526 comprises data such as the zip code, address, and mobile numbers for the individual and the primary subscriber. The orders table 524 comprises data such as the encounter ID, encounter start date, and encounter end date.

After identifying the data in database 522, the process 500 includes checking to determine whether orders have surpassed their stop date and time at 528. If the orders have not surpassed their stop date and time, then no further action 532 is needed as the individual is still within the timeframe to continue to take the prescribed medication for proper medical purposes. However, if the answer to whether orders have passed the stop date and time is yes 534, then the zip code for the individual is identified and locations of one or more specific drug collection centers from the DEA are identified at 536. Three of the nearest drug collection centers 538 to the individual are determined based on the individuals zip code. As mentioned with respect to FIGS. 3 and 4, the zip code of the individual may be obtained from the database 522 from a person table 526. Following this, smart address text mapping 550 is utilized and a message 546 is created that includes the order details, patient number/member phone number, and the nearest three collection centers to the individual. The order details are identified from the database 522 from the orders table 534. After the message 546 is created, a SMS text message 548 comprising the order details, patient/member phone number, and nearest three collection centers is sent to a cell phone 552 of the individual to notify the individual as to when the prescription stop date has occurred and where the nearest drug collection centers are for drop off. In some embodiments, the SMS text message 548 may be sent to family members, guardians, caretakers or any other individual identified as relevant to managing the healthcare of the individual to notify them when the prescription stop date will occur so that they can facilitate disposal of the medications. This may occur in situations where the individual is not able to dispose of the medications (e.g. if the individual is unable to drive and dispose of the medications). In further embodiments, the SMS text message may be sent to both the individual and others such as other family members, guardians, or caretakers. By providing this information electronically, the process 500 provides more efficiency and makes the process of returning unused prescription medications that fall within the controlled substance category much easier. Additionally, by providing messages to family members, guardians, or caretakers, it will increase the probability that unused medications will be properly disposed of.

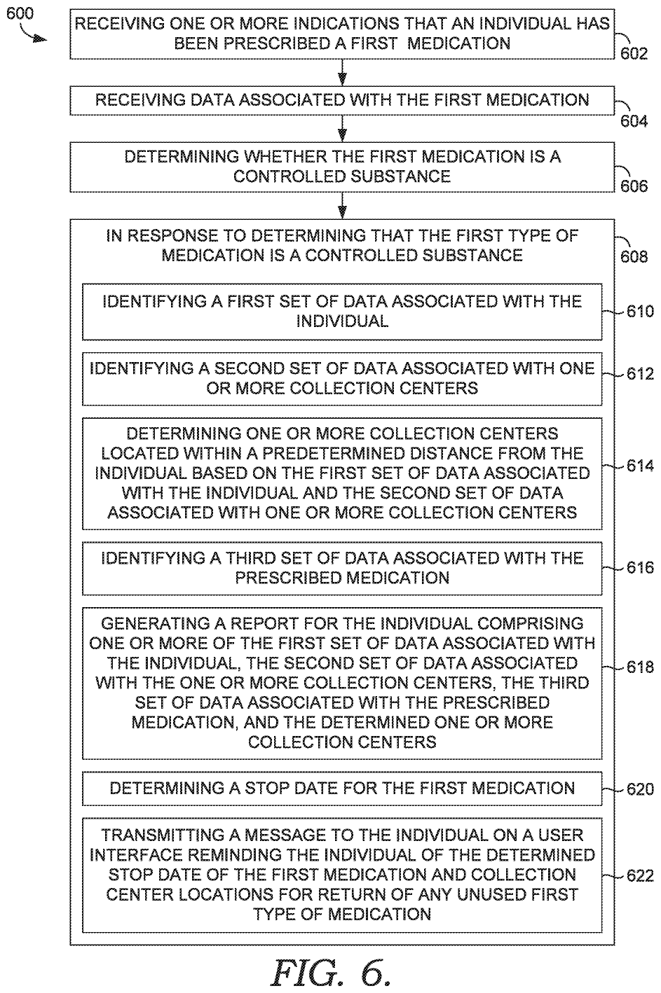

Next, FIG. 6 illustrates an exemplary method 600 for dynamically engaging with an individual and/or family members, caretakers, or guardians of the individual to increase return of unused controlled substance prescriptions to one or more collection centers, in accordance with aspects herein. Beginning with block 602, one or more indications that an individual has been prescribed a first medication is received by the indication receiver 212. Once the one or more indications are received, data associated with the first medication is received at 604 to determine whether the first medication is a controlled substance at 606. In response to determining that the first type of medication is a controlled substance at 608, a first set of data associated with the individual is identified by the first data set identifier 218. As previously discussed, the first set of data associated with the individual comprises biographical information such as the individual's zip code, address, and mobile phone number. Additionally, a second set of data associated with one or more collection centers is identified at 612 by the second data set identifier 220. The second set of data associated with the one or more collection centers includes the name and address of the collection centers identified. Based on the first set of data and second set of data, one or more collection centers located within a predetermined distance from the individual are determined at 614 by the collection center determiner 222. Once the one or more collection centers are determined, a third set of data associated with the prescribed medication is identified at block 616 by the third data set identifier 224. As discussed, the third set of data associated with the prescribed medication includes an encounter ID, orders start date and an order stop date. Following this, the generator 226 generates a report for the individual that comprises one or more of the first set of data associated with the individual, the second set of data associated with the one or more collection centers, the third set of data associated with the prescribed medication, and the determined one or more collection centers at block 618. Subsequent to generating the report for the individual at block 618, the stop date determiner 228 determines a stop date for the first medication at block 620. Once the stop date for the first medication is determined, a message is transmitted, by the transmitter 230, to the individual and/or family members, guardians, or caretakers on a user interface at block 622 that reminds the individual, family members, guardians, or caretakers of the determined stop date for the first medication and the one or more collection center location for return of any unused first type of medication.

Additionally, in some aspects, the transmitter 230 will send a second message to the individual and/or family members, guardians, and caretakers after the stop date has past or is about to occur to remind the individual to dispose of the unused medication at one or more of the collection centers identified. The second message transmitted to the individual may be transmitted after a predetermined time period has past. For example, the second message may be transmitted just prior to the order stop date (e.g. a day or week prior), on the order stop date itself, and then at one or more intervals after the order stop date has passed. When the order stop date has passed, additional messages may be transmitted, by the transmitter 220, to the individual to remind the individual that any unused medications should be returned to one of the collection centers determined and provided in the report generator by the generator 226.

It is further contemplated that, in aspects, the system 200 may have the capability of communicating with a collection center to determine whether unused medication has been dropped off after the order stop date has passed. When this occurs, the system may receive additional data that indicates that the unused first medication has been returned to the one or more collection centers. Once this occurs, the system 200 will stop sending messages to the individual reminding them to return the unused first medication. Such tracking of the unused medication may occur at the collection center by scanning of medication bottles with tracking IDs or potential tracking of actual medication pills that are dispensed. Any and all potential methods for tracking the return of the unused first medication and then subsequent communication between the collection center and system 200 are contemplated herein.

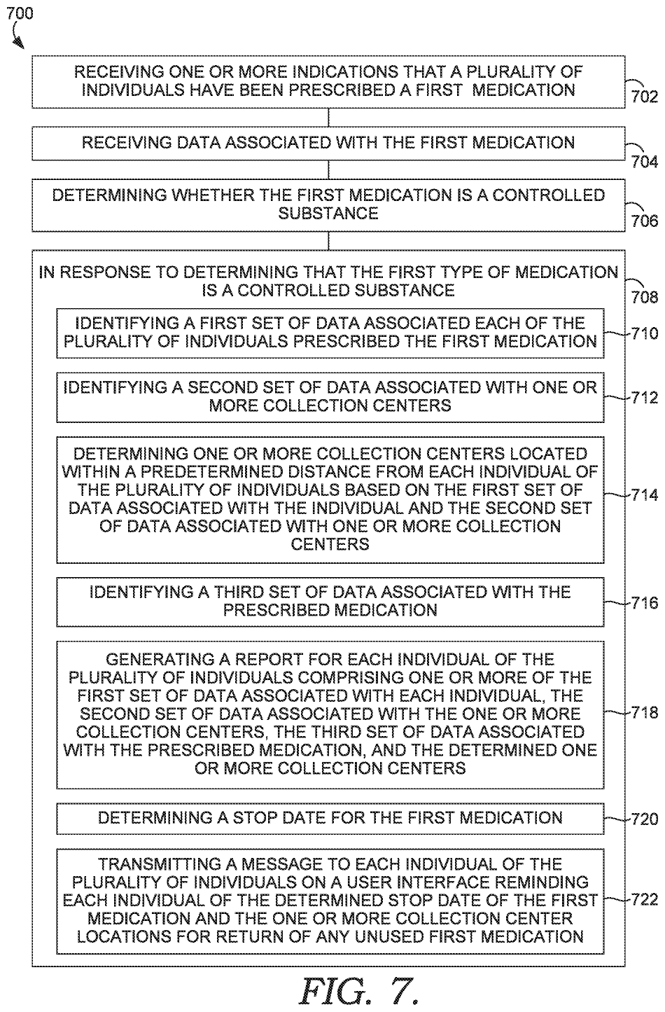

Moving on, FIG. 7 illustrates another exemplary method 700 for dynamically engaging with a plurality of individuals to increase return of unused controlled substance prescriptions to one or more collection centers. While FIG. 6 discussed engaging with a single individual to increase the return of unused controlled substances, it is contemplated that a similar method could be implemented for a plurality of individuals within a large patient population that are prescribed a first medication that is determined to be a controlled substance. This would be valuable as manually determining the location of each individual, whether each prescription for each individual is a controlled substance, and then determining one or more collection centers for drop off based on the individual's location for a whole patient population would be inefficient, costly, and not effective. As such, FIG. 7 illustrates a method in which the information for a plurality of individuals prescribed a first medication is processed at the same time to determine collection centers for drop off of unused medication for each individual.

Beginning with block 702, one or more indications that a plurality of individuals have been prescribed a first medication are received by indication receiver 212. Then at block 704, data associated with the first medication is received and then whether the first medication is a controlled substance is determined at 706 by the medication determiner 216. As previously described, the determination of whether the first medication is a controlled substance is made based on the data associated with the first medication received and by referencing a drug library that comprises the class categorization of medications and defines which medication are defined as controlled substances.

Next, at block 708, in response to determining that the first type of medication is a controlled substance, a first set of data associated with each of the plurality of individuals prescribed the first medication is identified at 712 by the first data set identifier 218. Similar to the process 600, the first set of data for each individual within the plurality of individuals includes each individual's zip code, address, full name, mobile number and other biographical information. Additionally, a second set of data associated with the one or more collection centers at block 712 is also identified by the second data set identifier 220. The second set of data includes the name and addresses of collection centers. Based on the identification of the first set of data and the second set of data, one or more collection centers located within a predetermined distance from each individual of the plurality of individuals is determined at block 714 by the collection center determiner 222.

After that, a third set of data associated with the prescribed medication is identified at step 716 by the third data set identifier 224. Then, the generator 226 will generate a report for each individual within the plurality of individuals that comprises one or more of the first set of data associated with each individual, the second set of data associated with the one or more collection centers, the third set of data associated with the prescribed medication, and the determined one or more collection centers at block 718. After the report is generated for the individual, a stop date for the first medication is determined at 720 by the stop date determiner 228 and a message is transmitted, by the transmitter 230, to each individual within the plurality of individuals on a user interface to remind each individual of the determined stop date of the first medication and the one or more collection center locations for return of any unused first medication at block 722.

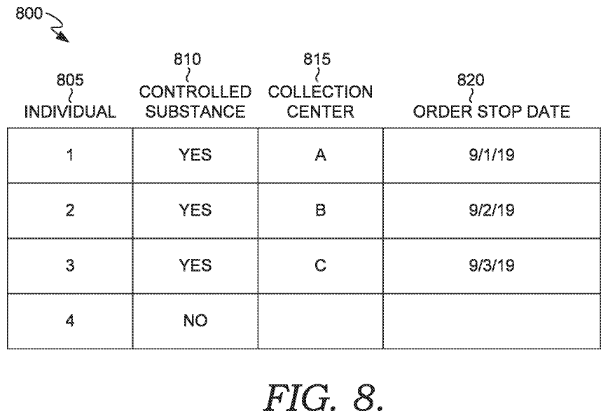

Next, FIG. 8 illustrates an exemplary chart 800 depicting how the method of FIG. 7 engages with a plurality of individuals to make determinations for each individual within the plurality of individuals 1-4. As discussed, when a large patient population is being managed, it may be difficult to individually employ the process of FIG. 6 to determine whether the first medication is a controlled substance and then provide the collection center information to each individual separately. Therefore, as described in FIG. 7, the data for a plurality of individuals prescribed a first medication is processed at the same time to determine collection centers for drop off of unused medication for each individual within the plurality of individuals. When process 700 described in FIG. 7 is complete, an exemplary chart 800 may be generated which depicts each individual within the plurality of individuals in column 805, whether the first medication prescribed to each individual is determined to be a controlled substance at 810, the determined collection center 815 for each individual, and the determined order stop date 820 for each first medication for each individual. For example, one or more indications that the plurality of individuals 1, 2, 3, and 4 have been prescribed a first medication and data associated with each of the first medications for each of the individuals of the plurality of individuals has been received. Based on the data received, the medication determiner 216 determines whether each first medication for each individual of the plurality of individuals 1-4 is or is not a controlled substance.

As shown in FIG. 8, the first medication for individuals 1-3 have been determined, via the medication determiner 216, to be a controlled substance. By contrast, the first medication for individual 4 has been determined not to be a controlled substance. As such, the process 700 will stop for the first medication for individual 4 and individual 4 can dispose of any remaining, unused, first medication in any manner without posing significant risk of misuse of the first medication. By contrast, in response to determining that the first medication is a controlled substance for individuals 1-3, the first data set identifier 218 will identify, as previously described, a first set of data associated with each of the plurality of individuals prescribed the first medication. Additionally, a second set of data associated with the one or more collection centers will be identified by the second data set identifier 220 as previously described.

Following this, the collection center determiner 222 will determine one or more collection centers located within a predetermined distance from each individual 1-3 based on the first set of data and the second set of data identified. As shown in the collection center column 815, a different collection center has been determined for each individual 1-3. For example, the collection center determiner 222 determined that collection center A is within a predetermined distance (e.g. within a 10 mile radius) from individual 1's zip code, thereby determining that collection center A should be the drop off location for any unused first medication by individual 1. Additionally, the collection center determiner 222 determined, based on identified individual zip codes, that the collection center for dropping of unused first medication by individual 2 is collection center B and the collection center for dropping off any unused first medication by individual 3 is collection center C. While FIG. 8 only shows one determined collection center 815 for each of the individuals 1-3, it is contemplated that in other aspects, more than one collection center 815 may be identified for each individual. In yet other aspects, three collection centers 815 may be determined for each individual, providing each individual with multiple location options to drop off the unused first medication. After identifying a third set of data, by the third data set identifier 224, that is associated with the first medication (e.g. the encounter ID and order details), the generator 115 will generate a report for each individual 1-3 that comprises the first set of data associated with each individual, the second set of set of data associated with the one or more collection centers, the third set of data associated with the prescribed first medication, and the determined one or more collection centers.

In addition to utilizing process 700 of FIG. 7 to determine whether the first medication prescribed to each individual of the plurality of individuals 1-4 is a controlled substance and the collection center for each substance, the stop date determiner 228 will also determine a stop date for each medication as shown at 820. In this example, the stop date shown for the first prescription for individual 1 is Sep. 1, 2019, for individual 2 is Sep. 2, 2019, and for individual 3 is Sep. 3, 2019. As previously discussed, the stop date determination may be based on identifying data associated with the first medication from an orders table 314 (shown in FIG. 3). Once the stop dates for each of the individuals 1-3 are determined by the stop date determiner 228, a message will be transmitted to each individual 1-3 by transmitter 230 reminding each individual 1-3 of the first medication's determined stop date and the one or more collection center locations (e.g. collection centers A-C) for drop off of any unused first medication after the determined stop date by individuals 1-3. Additionally, as previously mentioned, the message may also be transmitted to other individuals such as family members, guardians, and/or caretakers of the individual. The message transmitted may be transmitted prior to the stop date, on the date of the stop date, or after and may be transmitted to each individual via SMS text, email, on paper, or any other suitable communication method. Additionally, as previously mentioned, the transmitter 230 may also send subsequent messages reminding individuals 1-3 that the determined stop has passed in an effort to ensure that each individual drops off any remaining unused first medication to the determined collection center for each individual 1-3.

Embodiments described in the paragraphs above may be combined with one or more of the specifically described alternatives. In particular, an embodiment that is claimed may contain a reference, in the alternative, to more than one other embodiment. The embodiment that is claimed may specify a further limitation of the subject matter claimed.

The subject matter of embodiments of the invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

We claim:

1. A method for dynamically engaging with one or more of individuals and family members to increase return of unused controlled substance prescriptions to one or more collection centers, the method comprising:
receiving one or more indications that an individual has been prescribed a first medication;
receiving data associated with the first medication;
determining whether the first medication is a controlled substance;
in response to determining that the first type of medication is a controlled substance;
identifying a first set of data associated with the individual;
identifying a second set of data associated with one or more collection centers;
determining one or more collection centers located within a predetermined distance from the individual based on the first set of data associated with the individual and the second set of data associated with one or more collection centers;
identifying a third set of data associated with the prescribed first medication;
generating a report comprising one or more of the first set of data associated with the individual, the second set of data associated with the one or more collection centers, the third set of data associated with the prescribed medication, and the determined one or more collection centers;
determining a stop date for the first medication; and
transmitting a message to one or more of the individual and family members on a user interface reminding the individual of the determined stop date of the first medication and the one or more collection center locations for return of any unused first medication.

2. The method of claim 1, wherein the one or more indications that an individual has been prescribed a first medication is input into an electronic health record.

3. The method of claim 1, wherein the first medication is prescribed as an outpatient medication during an outpatient encounter with a healthcare provider.

4. The method of claim 1, wherein the first medication is prescribed as a part of discharge from a hospitalization.

5. The method of claim 1, wherein the first medication is a medication renewal.

6. The method of claim 1, wherein the first set of data associated with the individual comprises one or more of a zip code, address, mobile number, and member mobile number.

7. The method of claim 1, wherein the second set of data associated with the one or more collection centers comprises one or more of the collection center name, address, and distance from the individual.

8. The method of claim 1, wherein the third set of data associated with the prescribed medication comprises one or more of an encounter ID, order start date, and order stop date.

9. The method of claim 1, wherein the message transmitted to the individual and family members is a SMS text message.

10. The method of claim 1, wherein three collection centers are determined within the predetermined distance from the individual and provided to the individual in the generated report.

11. A system for dynamically engaging with an individual to increase return of unused controlled substance prescriptions to one or more collection centers, the system comprising:
one or more processors;
a storage device storing a computer program product comprising computer instructions that, upon execution by the one or more processors, cause the one or more processors to perform operations comprising:
receiving one or more indications that an individual has been prescribed a first medication;
receiving data associated with the first medication;
determining whether the first medication is a controlled substance;
in response to determining that the first type of medication is a controlled substance;
identifying a first set of data associated with the individual;
identifying a second set of data associated with one or more collection centers;
determining one or more collection centers located within a predetermined distance from the individual based on the first set of data associated with the individual and the second set of data associated with one or more collection centers;
identifying a third set of data associated with the prescribed medication;
generating a report for the individual comprising one or more of the first set of data associated with the individual, the second set of data associated with the one or more collection centers, the third set of data associated with the prescribed medication, and the determined one or more collection centers;
determining a stop date for the first medication; and
transmitting a first message to the individual on a user interface reminding the individual of the determined stop date of the first medication and the one or more collection center locations for return of any unused first medication.

12. The system of claim 11, wherein the second set of data associated with the one or more collection centers is identified based on data received from the Drug Enforcement Agency (DEA).

13. The system of claim 11, wherein the report generated for the individual is a part of an outpatient prescription summary.

14. The system of claim 11, wherein the report generated for the patient is a part of an inpatient discharge summary.

15. The system of claim 11, wherein the report generated for the patient is a part of a medication refill summary.

16. The system of claim 11, wherein the system transmits a second message to the individual after it has been determined that the unused first medication has not been returned to the one or more collection centers by the determined stop date.

17. The system of claim 11, wherein the system further receives data indicating that the unused first medication has been returned to the one or more collection centers.

18. A method for dynamically engaging with a plurality of individuals to increase return of unused controlled substance prescriptions to one or more collection centers, the method comprising:
receiving one or more indications that a plurality of individuals have been prescribed a first medication;
receiving data associated with the first medication;
determining whether the first medication is a controlled substance;
in response to determining that the first type of medication is a controlled substance;
identifying a first set of data associated with each of the plurality of individuals prescribed the first medication;
identifying a second set of data associated with one or more collection centers;
determining one or more collection centers located within a predetermined distance from each individual of the plurality of individuals based on the first set of data associated with the individual and the second set of data associated with one or more collection centers;
identifying a third set of data associated with the prescribed medication;
generating a report for each individual of the plurality of individuals comprising one or more of the first set of data associated with each individual, the second set of data associated with the one or more collection centers, the third set of data associated with the prescribed medication, and the determined one or more collection centers;
determining a stop date for the first medication; and
transmitting a message to each individual of the plurality of individuals on each individual's user interface reminding each individual of the determined stop date of the first medication and the one or more collection center locations specific to the individual for return of any unused first medication.

19. The method of claim 18, wherein the report is generated during a point of care.

20. The method of claim 19, wherein the point of care includes one or more of an outpatient prescription from an encounter with a healthcare provider, a medication reconciliation upon hospital discharge, and medication refills by a healthcare provider.
